(19) United States
(12) Patent Application Publication
(10) Pub. No.: US 2007/0214011 A1
(43) Pub. Date: Sep. 13, 2007

(54) Patient Discharge System and Associated Methods

(75) Inventor: Marc Demers, Winter Park, FL (US)

Correspondence Address: JACQUELINE E. HARTT, PH.D;ALLEN, DYER, DOPPELT, MILBRATH & GILCHRIST, P.A., P.O. BOX 3791, ORLANDO, FL, 32802-3791, US

(73) Assignee: HOSPITAL TRANSITIONS, LLC, Winter Park, FL (US)

(21) Appl. No.: 11/678,320

(22) Filed: Feb. 23, 2007

Related U.S. Application Data
(60) Provisional application No. 60/780,417, filed on Mar. 8, 2006.

(51) Int. Cl. G06Q 10/00; A61B 5/00

(52) U.S. Cl. 705/2; 600/300

(57) ABSTRACT

A system and method for improving patient discharge from a healthcare facility automates several areas of the patient discharge process, using automatically placed contacts with communications devices. The system includes a website that is accessible by registered users, primarily nurses and physicians. Critical elements of patient discharge, currently requiring many telephone calls between nurses and other personnel are converted to a largely automated “discharge cascade.” The cascade is initiated by the patient's nurse a predetermined period, for example, 24 hours, prior to a possible discharge, via the website. The website initiates an automatic communication with any or all involved physicians with a scripted, interactive call to elicit physician approval for the discharge. If the physician approves the discharge, a secondary set of automated communications are made to downstream parties, such as nursing operations, housekeeping, patient transport, home health, pharmacy, and patient family member.

### CROSS-REFERENCE TO RELATED APPLICATION

This application claims priority to provisional application Ser. No. 60/780,417, filed Mar. 8, 2006, entitled “Patient Discharge System and Associated Methods.”

### BACKGROUND OF THE INVENTION

1. Field of the Invention

The present invention relates to systems and methods for improving process flow within a healthcare facility, and, more particularly, to such systems and methods for improving a process associated with discharging patients from a hospital.

2. Description of Related Art

A shortage of hospital beds is a chronic problem for many hospitals, and can result in reduction in patient services, cancellation of elective, well-paying procedures, poor use of nurse and physician time, and emergency room crowding, among other difficulties. While more beds can be made available by building larger hospitals and hiring more staff, this is an expensive and usually long-range solution.

One of the chief bottlenecks in hospital patient processing is in the area of patient discharge, which in the present state of the art can require multiple telephone calls between nurses and physicians, nurses and pharmacists, nurses and ancillary personnel, and nurses and patient family members. Frequently the rate-limiting step in the process is the requirement for the physician to be physically present at the patient's bedside to authorize the patient's release. In addition, contacting a home health agency, when needed, is often done at the last minute and can result in additional delays.

Although there is known to exist a software package that can trigger an alert when a bed becomes available, there remains a need to increase the efficiency of the entire patient discharge process.

### SUMMARY OF THE INVENTION

It is therefore an object of the present invention to provide a system and method for improving patient discharge from a healthcare facility. The system and method automates several areas of the patient discharge process, using automatically placed contacts with communications devices. If hospital discharges can be accelerated to occur earlier in the business day, less manpower is needed, greater numbers of beds become available for patient use on a real-time basis, and revenue is increased.

The system includes a website that is accessible by registered users, primarily nurses and physicians. Critical elements of patient discharge, currently requiring many telephone calls between nurses and other personnel are converted to a largely automated “discharge cascade.” The cascade is initiated by the patient's nurse a predetermined period, for example, 24 hours, prior to a possible discharge, via the website. The website initiates an automatic communication with any or all involved physicians with a scripted, interactive call to elicit physician approval for the discharge.

If the physician approves the discharge, a secondary set of automated communications are made to downstream parties, such as nursing operations, housekeeping, patient transport, home health, pharmacy, and patient family member.

The features that characterize the invention, both as to organization and method of operation, together with further objects and advantages thereof, will be better understood from the following description used in conjunction with the accompanying drawing. It is to be expressly understood that the drawing is for the purpose of illustration and description and is not intended as a definition of the limits of the invention. These and other objects attained, and advantages offered, by the present invention will become more fully apparent as the description that now follows is read in conjunction with the accompanying drawing.

### BRIEF DESCRIPTION OF THE DRAWINGS

FIG. 1 is a system schematic of the patient discharge process of the present invention.

FIG. 2 is a communications flowchart of the patient discharge process.

FIG. 3 is a flowchart of the steps of the patient discharge process.

FIG. 4 is an exemplary screen displaying a patient list and associated data.

FIG. 5 is an exemplary screen displaying selection of the “orders” tab.

FIG. 6 is an exemplary screen displaying a list of discharge types.

FIG. 7 is an exemplary screen including a “new result” column.

FIG. 8 is an exemplary screen displaying active orders.

FIG. 9 is an exemplary screen displaying a list of responding physicians.

FIG. 10 is an exemplary screen displaying the most recent responding physician.

FIG. 11 is an exemplary screen displaying a complete list of responders.

FIG. 12 is an exemplary screen showing a negative result with a red flag.

FIG. 13 is an exemplary screen displaying a complete list of responders, including a negative responder.

### DETAILED DESCRIPTION OF THE PREFERRED EMBODIMENTS

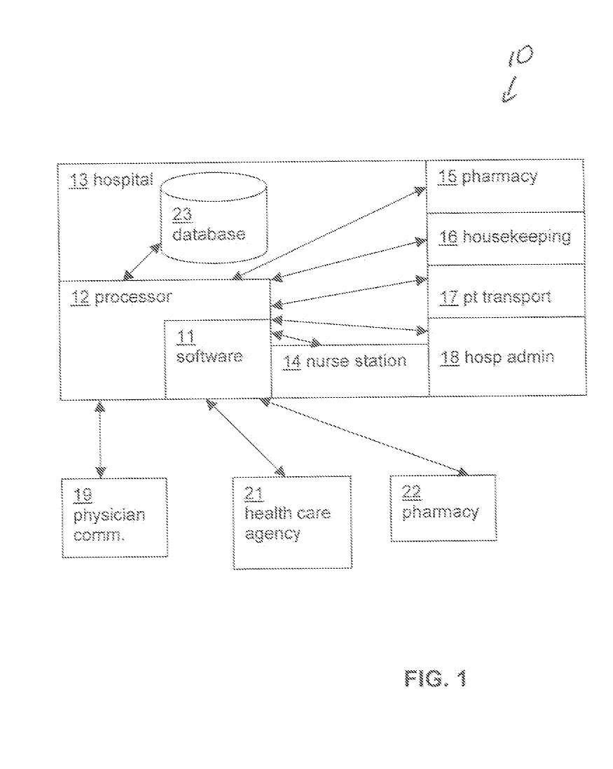

A description of the preferred embodiments of the present invention will now be presented with reference to FIGS. 1-13.

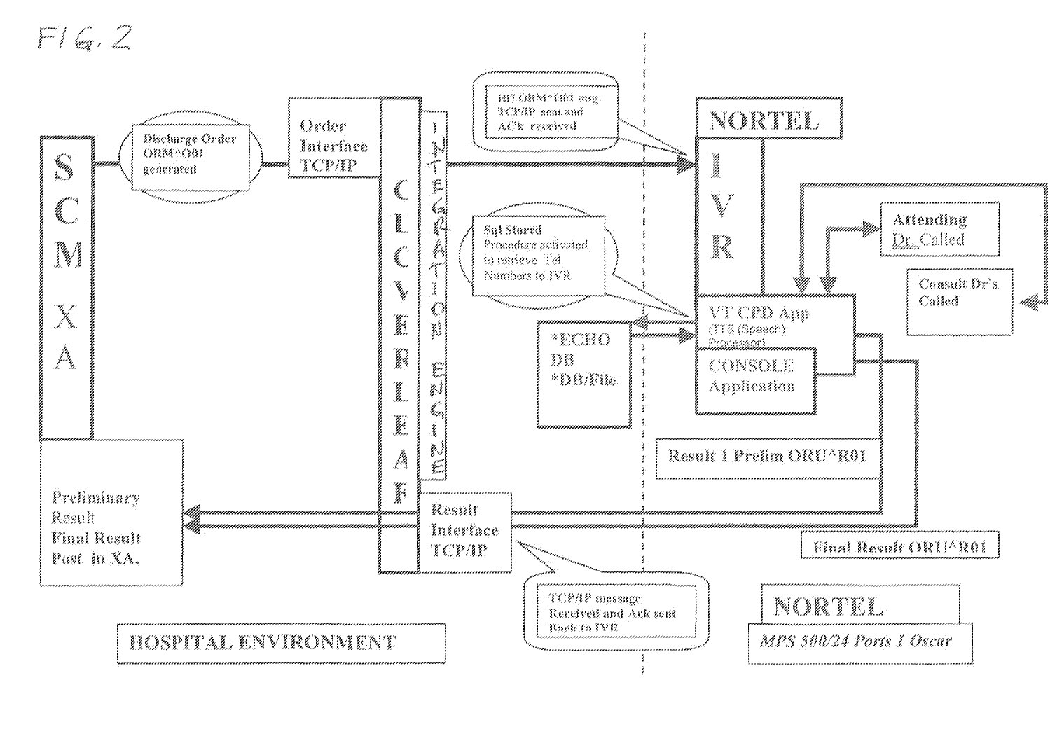

An exemplary embodiment of the system 10 (FIGS. 1 and 2) and method 100 (FIG. 3) are mediated by a software package 11 that is resident on a processor 12 that will typically be located at the hospital site 13, although this is not intended as a limitation. The processor 12 is in signal communication with a plurality of sites within the hospital 13, for example, workstations at nurses' stations 14, pharmacy 15, housekeeping 16, patient transport 17, and hospital administration 18. Each of the workstations 14-18 will require a secure login for access. The processor 12 is also adapted to establish signal communication with sites outside the hospital 13, such as physician communication devices 19 (cell phone, personal data assistant, personal computer, etc.), family member communication devices 20, home health care agency 21, and external pharmacy 22. The processor 12 is also in signal communication with a database 23 containing patient and physician data, such as contact information. In a particular embodiment, the system 10 functions over a service-oriented architecture for establishing secure communications over a plurality of communication pathways.

The software package 11 comprises a plurality of code segments that are adapted to mediate the relevant method steps of the invention. The method 100 (FIG. 3) includes the step of determining that a patient may be ready for discharge in a predetermined time period, for example, 24 hours (block 101). This determination is typically made by a nurse, although this is not intended as a limitation. The nurse logs into the system 10 (block 102) and initiates the automated discharge process (block 103) by selecting from a list of patient names (block 104) and accessing discharge-related data (block 105) such as the name and contact information for: the patient's admitting physician, any consulting physicians involved in the patient's care, a responsible family member or other party who will retrieve the patient, the patient's chosen pharmacy, and a home health agency (selected on the basis of patient insurance company and geographic location, by, for example, zip code). The patient's current medication list is also accessed, and can serve as a draft of a patient discharge medication list for the discharging physician(s).

Typically the data on the patient's admitting physician, responsible family member, pharmacy, and home health agency are available as of the admission process. The remaining data will likely change during the hospitalization.

The system 10 then automatically attempt to contact the admitting physician (block 106), for example, via his/her cell phone. A prerecorded message is sent by the system 10 (block 107), with an interactive script (block 108), for example: “Hello, Doctor (doctor's name).” The doctor must establish secure communication by speaking into his/her communication device (block 109), so that an interactive voice recognition (IVR) protocol can determine that the speaker is indeed the doctor being sought (block 110). If not, the system 10 terminates the call (block 111).

If the IVR determines that the correct person has been reached (block 110), the script continues (block 112) with a prompt such as: “The nurse (nurse's name) for your patient (patient name) has indicated that hospital discharge may be possible in 24 hours. If you agree, and wish to initiate the hospital discharge cascade, press 1. If you disagree, press 2. If you wish to speak with the nurse, press 3.” This last option initiates a call to the nurse (block 113).

If the physician presses 1, an interactive, autodialed prerecorded call is sent to all consulting physicians (block 114), with a similar script. This doctor must also establish secure communication by speaking into his/her communication device (block 115), so that the IVR protocol can determine that the speaker is indeed the doctor being sought (block 116). If not, the system 10 terminates the call (block 117). If the consulting physician has been reached correctly (block 116), the system 10 then continues with a script (block 118) such as: “The admitting physician, Dr. (admitting physician name) has indicated that his/her patient (patient name) will be ready for discharge in 24 hours. If you agree with this hospital discharge, press 1. If you wish discharge to be withheld, press 2.”

If either the admitting or consulting physician presses 2, the system 10 is so updated, and the discharge cascade is terminated (block 119). If the admitting and all the consulting physician(s) press 1, the discharging physicians are required to review and alter as necessary the patient's medication list (block 120).

The discharge cascade continues by initiating a plurality of communications, including autodialed calls, pages, emails, and/or faxes. For example, within the hospital 13, prerecorded messages can be sent to nursing operations 14, patient transport 17, social work, etc. (block 121). These calls can be initiated at any time respectively deemed appropriate during the discharge sequence. Communications outside the hospital 13 can include calls to the patient's responsible relative or other responsible party 20, informing them of the patient's possible discharge (block 122), and of the time of pickup. If appropriate, the home health agency 21 is also notified (block 123), as well as the patient's pharmacy 22, transmitting a list of patient prescriptions (block 124) as mandated by the discharging physician(s) in block 118. At the appointed time, the patient is discharged (block 125), and the hospital bed is vacated, which initiates housekeeping activities, etc., prior to bed re-use (block 126).

Preferably a reporting function is included in the system 10, wherein all contacts are recorded (time and party), and when discharge-related activities were carried out by that party. Such reporting can serve several purposes, including providing data that can be analyzed for the purpose of achieving process improvement and for providing an audit function.

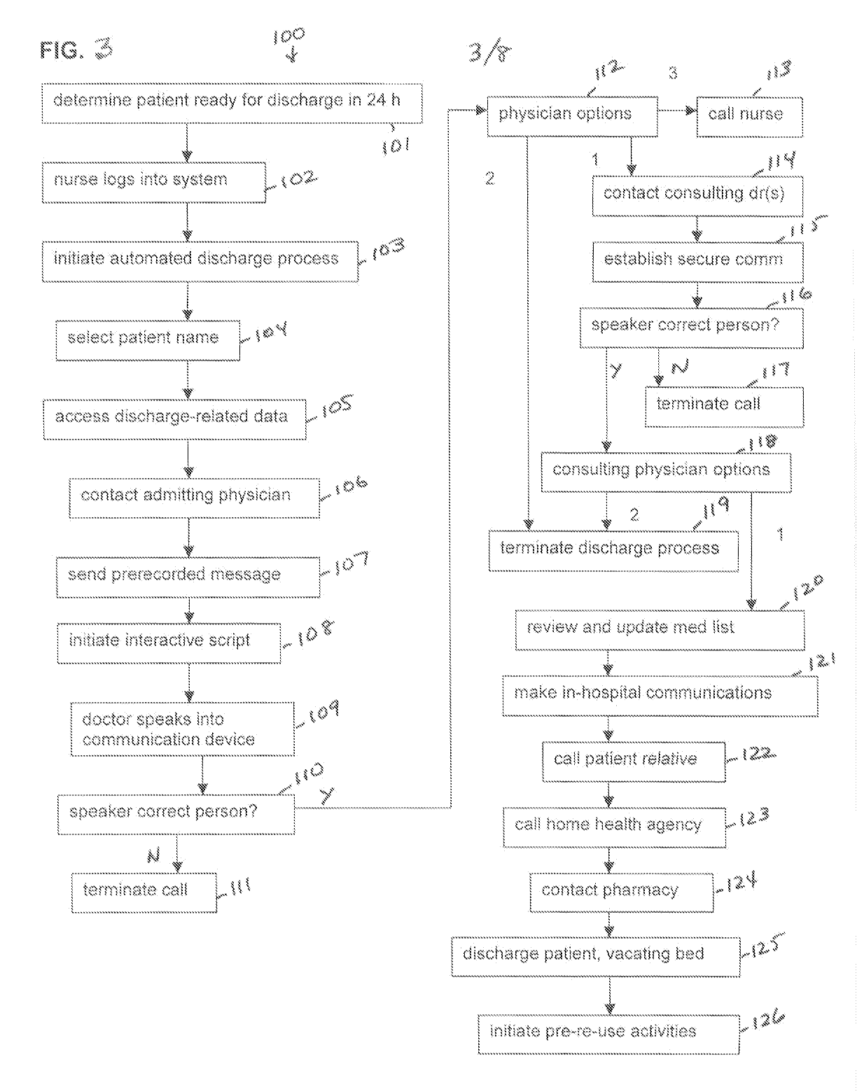

An exemplary set of screens (FIGS. 4-13) illustrates the steps taken by the hospital-site user. After a secure log-in, the system 10 brings up a patient list on a screen 50 such as shown in FIG. 4. On the list are shown the assigned location 51 for each patient 52, as well as ID information 53, visit status 54, gender 55, age 56, healthcare provider 57, and reason for visit 58. On the far left is a “check orders” column 59 with indicators such as a green flag 60 or a red flag 61 with an exclamation point thereon.

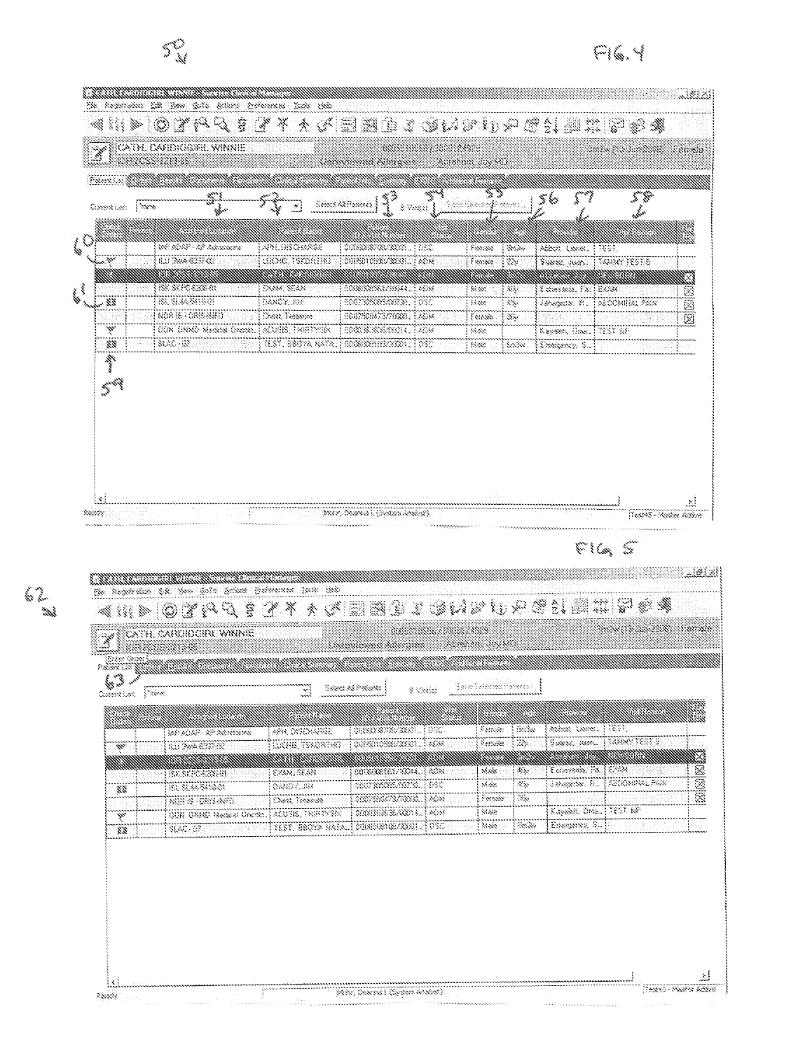

In the next screen 62 (FIG. 5), the user has selected the “orders” tab 63, and entered “discharge.” The screen 64 of FIG. 6 provides a list of discharge types; here, the “discharge tomorrow—request approval” item 65 is selected, which initiates the discharge cascade for the selected patient.

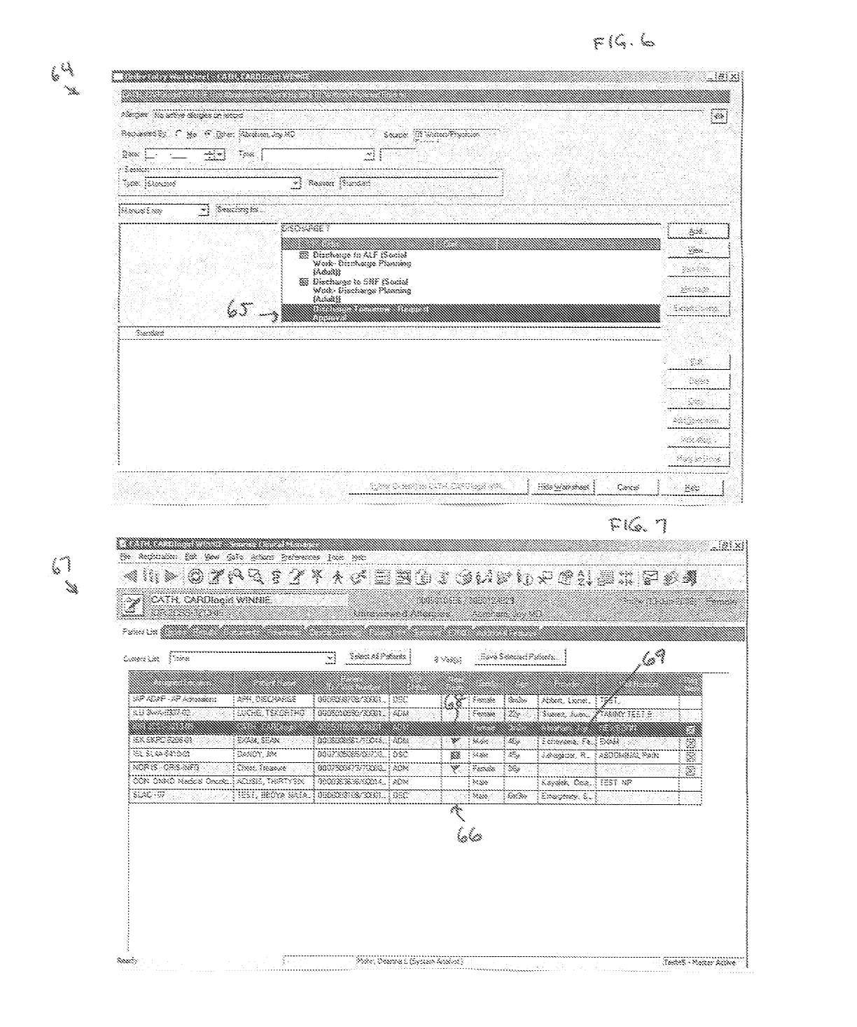

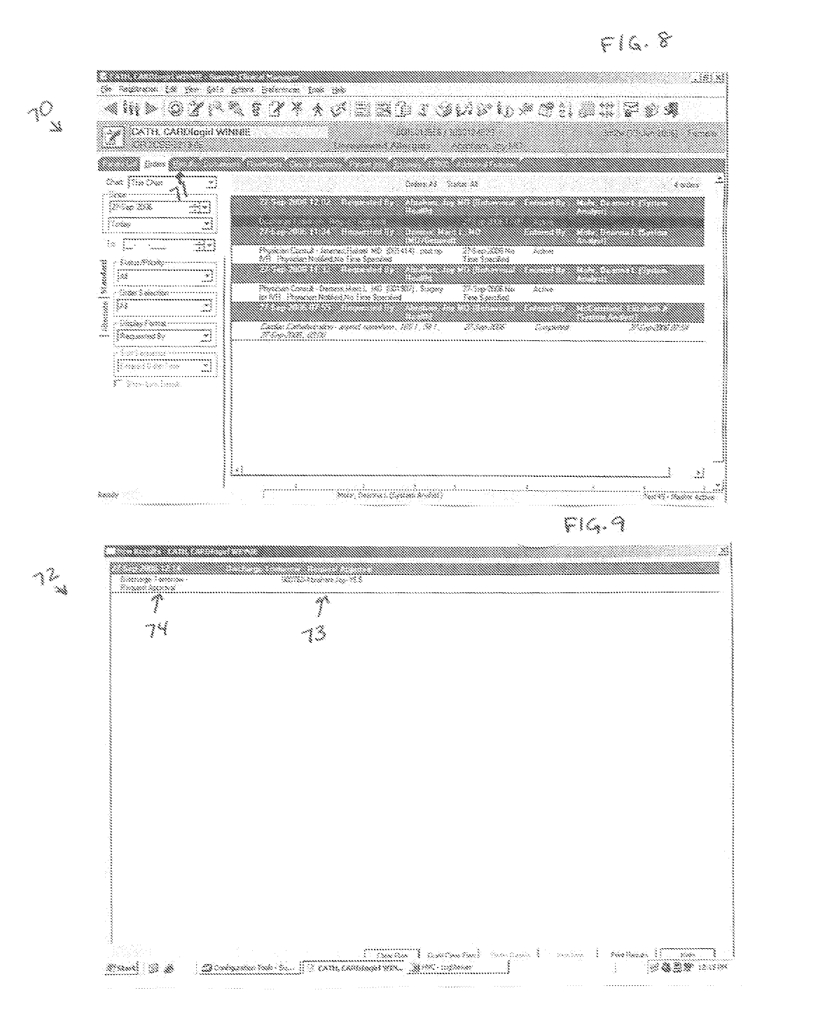

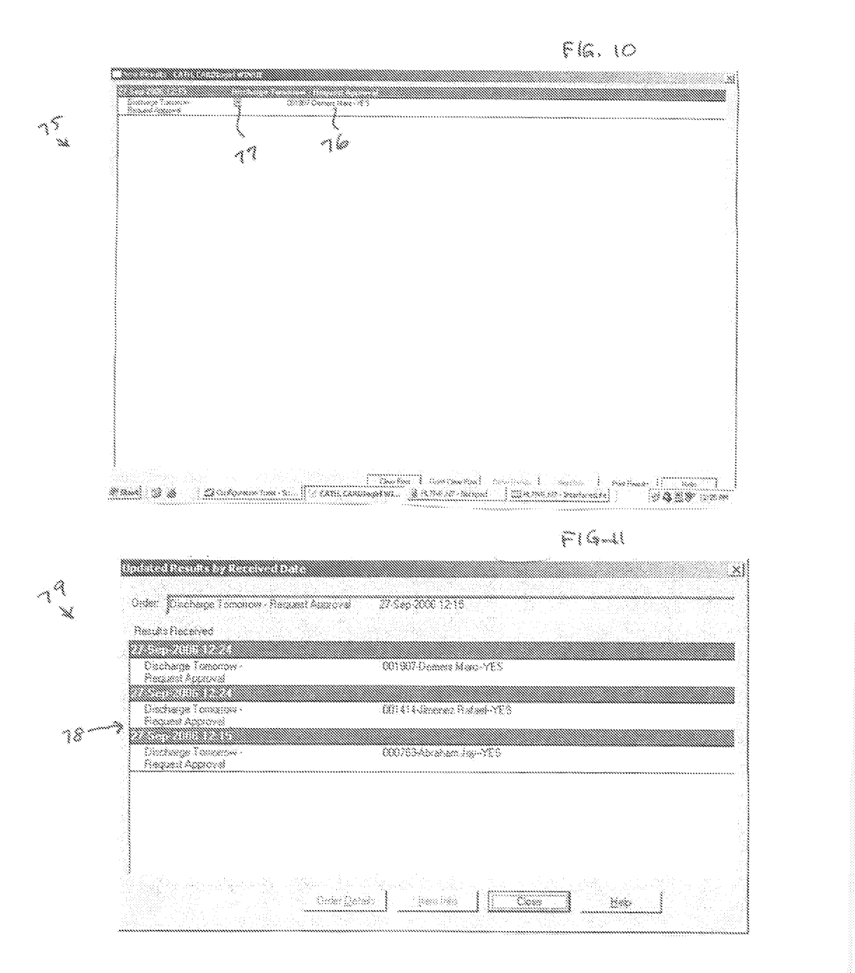

When the doctor responds via the IVR system, a “new result” column 66 appears on the screen 67 of FIG. 7, here a green flag 68, indicating that the contacted doctor Abraham 69 has approved the discharge. If this sequence has occurred during business hours, the order becomes active, as shown in the screen 70 of FIG. 8. Selecting the “Results” tab 71 brings up the screen 72 of FIG. 9, which displays a list 73 of the responding physicians and their responses to the query 74 in the first column. This screen 72 can also be called up by double-clicking on the flag 68 in FIG. 7. The screen 75 of FIG. 10 displays the most recent physician 76 responding, and clicking on the “H” 77 brings up a complete list 78 of responders on the screen 79 of FIG. 11.

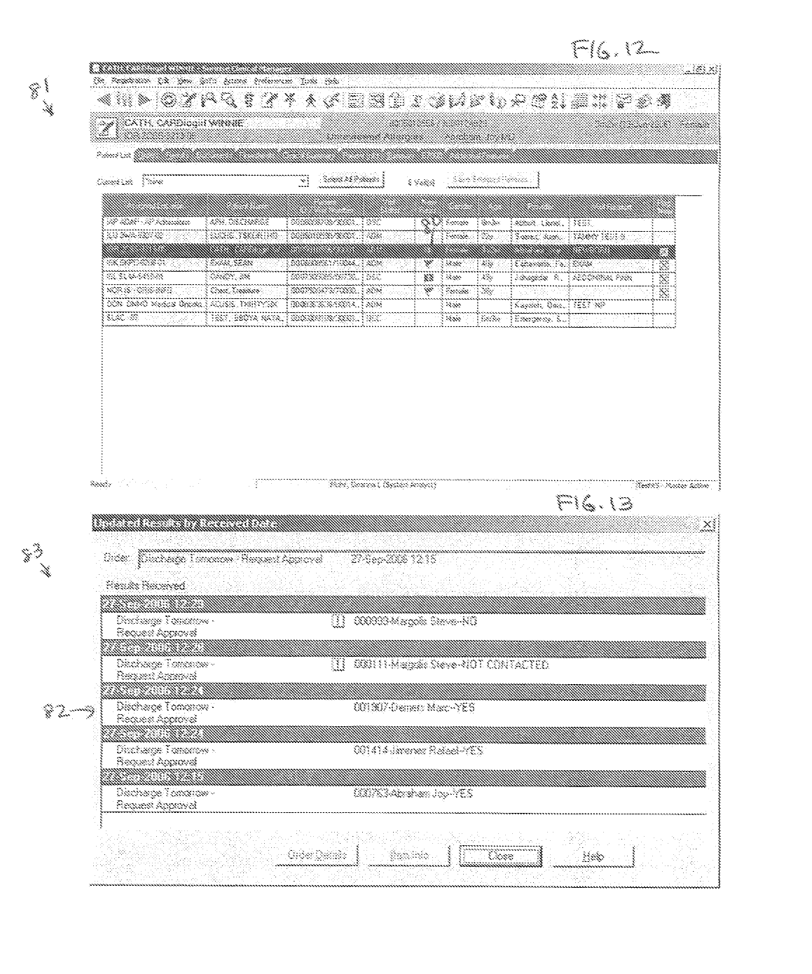

If a physician responds negatively, or cannot be contacted, the “new results” flag 80 on the screen 81 of FIG. 12 is red. Selecting that flag 80 again brings up the list 82 of responders in the screen 83 FIG. 13, showing that, when Dr. Margolis was contacted, he recommended not releasing the patient on the next day.

The system 10 and method 100 of the present invention measurably shift hospital discharge times into an earlier part of the day, creating greater bed availability. The workload of nurses and physicians is reduced by automating the redundant but necessary components of discharge, freeing them for direct patient care. This is achieved by permitting many of the process elements to occur in parallel, and by receiving physician instructions remotely, rather than waiting for the physician to appear at the patient's bedside. Thus discharge can become a gradual process, occurring over the entire day and at the physician's convenience. The physician is motivated to provide timely discharge in direct exchange for better use of his/her time and for increased freedom of action. The hospital benefits by obtaining use of the released bed earlier, and using staff more effectively. The patient benefits by being released in a timely fashion.

It is to be understood that the present invention is not to be limited to the above exemplary embodiment, and that extension to other healthcare situations such as transfer between hospital departments (into and out of critical care, for example) can also benefit from such a system.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the system and method illustrated and described herein are by way of example, and the scope of the invention is not limited to the exact details of implementation.

Having now described the invention, the system, the operation and use of preferred embodiments thereof, and the advantageous new and useful results obtained thereby, the new and useful configurations, and reasonable equivalents thereof obvious to those skilled in the art, are set forth in the appended claims.

What is claimed is:

1. A method for improving an efficiency of discharging a patient from a healthcare facility comprising the steps of:
initiating a communication with a physician remote from a patient requesting approval to discharge the patient from a healthcare facility within a predetermined time;
receiving a communication from the physician including an approval to discharge the patient from the healthcare facility within the predetermined time;
receiving from the physician, if desired, an order for an update to a medication protocol for the patient; and
automatically communicating physician approval to at least one healthcare facility department for implementing patient discharge within the predetermined time.

2. The method recited in claim 1, wherein the initiating step comprises initiating a telephone call to a physician telephonic device, and further comprising the steps of:
prompting the physician to utter a spoken message into the telephonic device;
analyzing the spoken message via a voice recognition protocol to ensure that the spoken message was uttered by the physician intended to be called;
if the analysis indicates that the spoken message is not from the intended physician, terminating the telephone call; and
if the analysis indicates that the spoken message is from the intended physician, proceeding to the communication-receiving step.

3. The method recited in claim 1, further comprising the steps, following the communication-initiating step, of offering the physician an option to:
contact a nurse directly;
terminate the discharge process; and
proceed to the communication-receiving step.

4. The method recited in claim 1, wherein the physician comprises a primary physician, and further comprising the steps, following the communication-receiving step, of:
contacting a consulting physician different from the primary physician, the consulting physician remote from the patient;
receiving a communication from the consulting physician including an approval to discharge the patient from the healthcare facility within the predetermined time;
receiving from the consulting physician, if desired, an order for an update to a medication protocol for the patient; and
automatically communicating consulting physician approval to the at least one healthcare department for implementing discharge within the predetermined time.

5. The method recited in claim 4, wherein the consulting-physician-contacting step comprises initiating a telephone call to a consulting physician telephonic device, and further comprising the steps of:
prompting the consulting physician to utter a spoken message into the telephonic device;
analyzing the spoken message via a voice recognition protocol to ensure that the spoken message was uttered by the consulting physician intended to be called;
if the analysis indicates that the spoken message is not from the intended consulting physician, terminating the telephone call; and
if the analysis indicates that the spoken message is from the intended consulting physician, proceeding to the communication-receiving step.

6. The method recited in claim 4, further comprising the steps, following the consulting-physician-contacting step, of offering the consulting physician an option to:
contact a nurse directly;
terminate the discharge process; and
proceed to the step of receiving a communication from the consulting physician.

7. The method recited in claim 1, wherein the physician-approval-communicating step comprises contacting at least one of nursing operations, patient transport, and social work department.

8. The method recited in claim 1, further comprising the step, following the physician-approval-communicating step, of automatically initiating contact with an extra-healthcare-facility site for communicating data on the patient discharge.

9. The method recited in claim 8, wherein the extra-healthcare-facility site comprises at least one of a patient relative/responsible party, a home health agency, and a pharmacy.

10. The method recited in claim 1, further comprising the steps, following the physician-approval-communicating step, of discharging the patient and initiating activities attendant upon re-use of a hospital space being vacated by the patient.

11. A system for improving an efficiency of discharging a patient from a healthcare facility comprising:
a healthcare facility communication device adapted to transmit a signal to, and receive a signal from, a physician communication device remote therefrom;
a processor in signal communication with the healthcare facility communication device and having software resident thereon comprising:
a code segment for initiating a communication from the healthcare facility communication device to the physician communication device requesting approval to discharge a patient from a healthcare facility within a predetermined time;
a code segment for receiving a communication from the physician communication device via the healthcare facility communication device including an approval to discharge the patient from the healthcare facility within the predetermined time;
a code segment for receiving from the physician communication device via the healthcare facility communication device an order for an update to a medication protocol for the patient; and
a code segment for automatically communicating physician approval to at least one healthcare facility department for implementing patient discharge within the predetermined time.

12. The system recited in claim 11, wherein the physician communication device comprises a telephonic device, and the initiating code segment comprises initiating a telephone call to the physician telephonic device, and wherein the software further comprises code segments adapted to:
prompt the physician to utter a spoken message into the telephonic device;
analyze the spoken message via a voice recognition protocol to ensure that the spoken message was uttered by the physician intended to be called;
if the analysis indicates that the spoken message is not from the intended physician, terminate the telephone call; and
if the analysis indicates that the spoken message is from the intended physician, proceed to the communication-receiving code segment.

13. The system recited in claim 11, wherein the software further comprises code segments adapted to, following the communication-initiating code segment, of offer the physician an option to:
contact a nurse directly;
terminate the discharge process; and
proceed to the communication-receiving step.

14. The system recited in claim 11, wherein the physician comprises a primary physician, the healthcare facility communication device is further adapted to transmit a signal to, and receive a signal from, a consulting physician communication device remote therefrom and wherein the software further comprises code segments adapted to, following the communication-receiving code segment, of:
contact a consulting physician different from the primary physician, the consulting physician remote from the patient;
receive a communication from the consulting physician including an approval to discharge the patient from the healthcare facility within the predetermined time;
receive from the consulting physician, if desired, an order for an update to a medication protocol for the patient; and
automatically communicate consulting physician approval to the at least one healthcare department for implementing discharge within the predetermined time.

15. The system recited in claim 14, wherein the consulting-physician-contacting code segment comprises a code segment for initiating a telephone call to a consulting physician telephonic device, and wherein the software further comprises code segments adapted to:
prompt the consulting physician to utter a spoken message into the telephonic device;
analyze the spoken message via a voice recognition protocol to ensure that the spoken message was uttered by the consulting physician intended to be called;
if the analysis indicates that the spoken message is not from the intended consulting physician, terminate the telephone call; and
if the analysis indicates that the spoken message is from the intended consulting physician, proceed to the communication-receiving step.

16. The system recited in claim 14, wherein the software further comprises code segments, following the consulting-physician-contacting code segment, adapted to offer the consulting physician an option to:
contact a nurse directly;
terminate the discharge process; and
proceed to the step of receiving a communication from the consulting physician.

17. The system recited in claim 11, wherein the physician-approval-communicating code segment is adapted to contact at least one of nursing operations, patient transport, and social work department.

18. The system recited in claim 11, wherein the software further comprises a code segment adapted to, following the physician-approval-communicating code segment, automatically initiate contact with an extra-healthcare-facility site for communicating data on the patient discharge.

19. The system recited in claim 18, wherein the extra-healthcare-facility site comprises at least one of a patient relative/responsible party, a home health agency, and a pharmacy.

20. The system recited in claim 11, wherein the software further comprises a code segment, following the physician-approval-communicating code segment, of communicating with at least one department in the healthcare facility to initiate activities attendant upon re-use of a hospital space being vacated by the patient.
